(19) United States
(12) Patent Application Publication
(10) Pub. No.: US 2009/0214640 A1
(43) Pub. Date: Aug. 27, 2009

(54) DELAYED RELEASE PHARMACEUTICAL ORAL DOSAGE FORM AND METHOD OF MAKING SAME

(75) Inventors: Pompilia Szabo, Greenfield Park (CA); Horst Zerbe, Hudson (CA)

Correspondence Address: FULBRIGHT & JAWORSKI L.L.P., 600 CONGRESS AVE., SUITE 2400, AUSTIN, TX, 78701, US

(73) Assignee: Intelgenx Cporp.

(21) Appl. No.: 11/817,721

(22) PCT Filed: Mar. 3, 2006
(86) PCT No.: PCT/CA06/00336

§ 371 Date: Aug. 20, 2008

Related U.S. Application Data
(60) Provisional application No. 60/658,182, filed on Mar. 4, 2005.
(60) Provisional application No. 60/671,523, filed on Apr. 15, 2005.

(51) Int. Cl. A61K 9/52; A61K 9/26; A61K 9/16; A61K 47/32

(52) U.S. Cl. 424/452; 424/465; 424/470; 424/458; 424/501; 514/772.6

(57) ABSTRACT

The present invention relates to a delayed release pharmaceutical oral dosage form and method of making same. The delayed release dosage form comprises one or more active ingredients within a granulated composition, which further comprises one or more excipients selected from the group of solid aliphatic alcohols, fatty acid esters, mixtures of esters of saturated fatty alcohols and saturated fatty acids, natural waxes, synthetic waxes, hydrogenated castor oil, hydrogenated vegetable oil, gums, and mixtures thereof; and one or more polymers or copolymers exhibiting a pH-dependent solubility. The present invention also related to method of making these delayed release dosage form.

### FIELD OF THE INVENTION

The present invention relates to a delayed release pharmaceutical oral dosage form and method of making same.

### BACKGROUND OF THE INVENTION

The prior art teaches the use of enteric film coatings on tablet's cores containing an active ingredient to delay the release of the active ingredient and therefore provide delayed release pharmaceutical oral dosage forms.

Enteric film coatings are used to allow the active ingredient(s) in a pharmaceutical oral dosage forms to be released in the intestine rather than in the stomach. Indeed, such active ingredients are often better absorbed via the intestine. Also, many pharmaceutical products may irritate the stomach. Others undergo chemical changes in gastric acid or by the action of stomach or saliva enzymes, and may thereby become less effective.

Enteric coatings are generally pH-sensitive coatings that will remain essentially impermeable at lower pH so as to pass through the stomach unscathed. Once in a higher pH region of the digestive tract, namely the intestinal tract, the coating will become permeable and allow the release of the active ingredients. Examples of such enteric coatings include the very well known coatings sold by Rohm Pharma under the trademarks Eudragit®. These coatings exist in various grades.

However, the use of enteric coatings involves additional costs and their formulation and application require skill and know-how. Furthermore, additional excipients such as plasticizers, glidants, anti-foaming agents, fillers and pigments are usually required to obtain suitable coating properties. For example, a coating must not be too thick, too brittle, too thin, too sticky, etc. All these parameters introduce technical and cost factors in the manufacturing of enterically coated dosage forms.

Various known techniques can be used to apply enteric coatings. Dry coating, spray coating and pan coating are just a few examples. Enteric coatings are commonly applied to compressed tablet core or to individual beads or pellets, which are then compressed into a tablet or placed in a reservoir capsule.

When the enteric coatings are applied to a compressed tablet core, the procedure generally consists in the preparation of an aqueous dispersion/solution or an organic solution, which includes a polymer providing the enteric release of the active ingredient and plasticizers, glidants, anti-foam agents, fillers and pigments. The polymer providing the enteric release of the active ingredient is generally a cellulose derivative or a polymer or a copolymer of acrylic and/or methacrylic acids or esters thereof, etc. In the case of methacrylic polymers, the coating is usually sprayed onto rotating tablets pre-warmed to about 30° C. and maintained at a temperature of approx. 25° to 35° C. during the entire process. A post-drying step, also known as tablet curing, contributes to film coalescence and improves the characteristics of the film coating.

Consequently, a new alternative for providing oral dosage forms which can essentially behave as an enterically coated dosage forms without the need for the formulation and application of an enteric coating would be a welcome innovation in the pharmaceutical art.

Prior art on the subject of delayed release is often concerned with non-steroidal anti-inflammatory drug (“NSAID”) formulations.

NSAIDs are among the most commonly prescribed and used drugs world-wide. Despite their therapeutic benefits, their use is associated with a reported increased risk of gastrointestinal side-effects, such as peptic ulceration, dyspeptic symptoms, risk of bleeding and perforation of the stomach (McGarty D M, Gastroentorology 1989, 96, 662; Hawkey C, BMJ 1990, 300, 278).

Promising solutions for the treatment and prevention of the gastrointestinal side-effects associated with the prolonged use of NSAIDs are:
to avoid contact between the NSAID and the acidic gastric juices by delaying the NSAID release, so that the NSAID is mainly released in the intestine rather that in the stomach, and
to combine the NSAID treatment with an anti-ulcer drug, such as prostaglandin analogues, H₂-receptor antagonists or proton pump inhibitors.

U.S. Pat. No. 5,698,225 proposes a combined NSAID and prostaglandin product. The product is enterically coated and is composed of a core, comprising an NSAID selected from diclofenac and piroxicam, surrounded by a mantle coating comprising a prostaglandin. An intermediate coating can be present between the NSAID core and prostaglandin mantle coating.

Other methods describe the formation of coated granules by spraying a solution of a methacrylic copolymer (e.g. Eudragit®) onto a bed of NSAID or other drug and any necessary excipients using, for example, a fluid bed coating apparatus (U.S. Pat. No. 6,537,582). This process is controlled so as to produce fine beads that do not require milling before incorporation into tablets or capsules. These beads may include cellulose derivatives (e.g. hydroxypropyl methyl cellulose) and methacrylic acid and its derivatives (e.g. methyl methacrylates, for example: Eudragrit.RTM®, especially Eudragrit® L or S). Normally the coating will include plasticizers, such as polyethylene glycol, triacetin or phthalate esters, that confer required characteristics to the films.

U.S. Pat. No. 6,365,184 and published US Patent Application 2004/0022846A1 describe an oral pharmaceutical dosage form comprising an acid susceptible proton pump inhibitor and one or more NSAIDs in a fixed formulation, wherein the proton pump inhibitor is protected by an enteric coating layer. The fixed formulation is in the form of an enteric coated layered tablet, a capsule or a multiple unit tableted dosage form.

U.S. Pat. No. 6,287,600 discloses a procedure to stabilize a pharmaceutical composition which includes a NSAID. In manufacturing, the NSAID, e.g. diclofenac sodium, is first granulated by blending it with acceptable ingredients (diluents, binders) in a fluid-bed granulator. The granules so obtained are enterically coated with an enteric dispersion containing methacrylic acid copolymer type C, NF, triacetin, and antifoam 1520-US. The enterically coated diclofenac granules are then blended with microcrystalline cellulose PH 102 and hydrogenated castor oil powder.

As is apparent from above, enterically coated systems involve various technical parameters which are time-consuming and which increase manufacturing costs. Coating ingredient selection, dispersion preparations and various technical parameters (i.e. temperature range, droplet size, type and content of plasticizer, etc.) are time-consuming operations and furthermore, the application of many layers of coating (i.e. undercoatings) is often necessary to obtain an efficient protection.

Enteric coating performance, brittleness and stickiness underline critical shortcomings of enterically coated systems. The present invention aims to provide an improvement over the prior art by providing preparations which do not require enteric coating and yet behave as enterically coated products.

Granulating techniques are also known in the art. Various processes are well known in the pharmaceutical art for modifying starting powders or other particulate materials. In these processes, the powders are typically mixed together with a binder material into larger permanent free-flowing agglomerates or granules referred as a “granulation.” For example, solvent-using “wet” granulation processes are generally characterized in that the powders are combined with a binder material and moistened with water or an organic solvent under conditions allowing the formation of a wet granulated mass from which the solvent must then be evaporated.

Melt granulation techniques have also been developed in the art and generally consist in the use of room temperature solid or semi-solid materials (having a relatively low softening or melting range) to promote granulation of powdered or other materials, essentially in the absence of added water or other liquid solvents. The room temperature solid or semi-solid materials, when heated to a temperature within their melting range, liquefy to act as a binder or granulating medium. This liquid spreads over the surface of powdered materials with which it is associated, and on cooling, forms a solid granulated mass in which the initial materials are bound together. The resulting granules can then be pressed into tablets or encapsulated.

U.S. Pat. No. 5,169,645 describes a wax-containing composition having improved flow properties. These properties are obtained by admixing waxes in the melt with certain flow improving additives, followed by cooling and granulation of the admixture. In certain embodiments of the invention, only the wax itself melts in the melt combination of the wax(es) and additives(s), and in other cases both the wax(es) and the additives(s) will both melt. In either case, the melt combination of the wax(es) with the additive(s) yields, upon cooling and granulation, a wax-containing particulate drug diluent having improved flow properties. The wax-containing granules are directly compressible but are not used to delay or control drug release, indeed their only stated purpose is to improve the flow properties.

Published US patent application 2003/0068363 provides a tablet obtained by direct compression. The tablet comprises at least 60 weight % of an active ingredient and a powdered wax having a melting point greater than about 90° C. This composition provides an immediate release of the active ingredient. The wax is selected from the group consisting of linear hydrocarbons, microcrystalline wax, and mixtures thereof. The tablet is substantially free of water-soluble, non-saccharide polymeric binders and comprises at least one outer coating. The outer coating comprises a material selected from the group consisting of gelatin, isomalt, monosaccharides, disaccharides, polysaccharides (such as starch), cellulose derivatives, shellacs, and polyhedric alcohols (such as xylitol, mannitol, sorbitol, maltitol, erythritol, and polyalkylene glycols). The tablet contains the active ingredient, which may be a analgesic, in its native crystalline form.

Clearly, the prior art has thus far failed to consider or apply a granulation technique, for example a melt-granulation technique, using as part of the ingredient mix a pH-dependant material which will make the product behave as an enterically coated product.

Another drawback of current pharmaceutical products is light-induced degradation of active ingredients. The traditional approach to remedy this problem has been to coat the solid dosage forms with coatings containing opacifying pigments, such as titanium dioxide white. These coatings commonly comprise polymers and additives, which facilitate their application and provide good mechanical resistance. It is also known to mix pigments with active and inactive powder excipients.

U.S. Pat. No. 4,900,557 provides a sustained release coated pellet formulation with titanium dioxide, BaSO₄, iron oxide and ferrum reductum being used as weighting agents. However, heretofore, light-protecting pigments have not been suggested nor used in melt-granulation so as to impart light-protective properties to the resulting granules.

### SUMMARY OF THE INVENTION

A delayed release oral dosage form comprising one or more active ingredients within a granulated composition, which further comprises one or more excipients selected from the group of solid aliphatic alcohols, fatty acid esters, mixtures of esters of saturated fatty alcohols and saturated fatty acids, natural waxes, synthetic waxes, hydrogenated castor oil, hydrogenated vegetable oil, gums, and mixtures thereof, and one or more polymers or copolymers exhibiting a pH-dependent solubility.

This delayed release oral dosage form can be a monolithic dosage form, such as a compressed tablet with or without a coating, wherein the granulated composition, along with a extra-granular excipient phase if present, is compressed into a monolithic shape or it can be a capsule containing the granulated composition and the extra-granular excipient phase.

In a specific embodiment, the one or more polymers or copolymers are acrylic and methacrylic acid polymers and copolymers such as acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

In another specific embodiment the one or more excipients is a fatty alcohol, a saturated fatty alcohol or a solid aliphatic alcohol.

In yet another specific embodiment the active ingredients is selected from an NSAID, a prostaglandin and mixtures thereof.

The delayed release oral dosage of the invention can further comprises one or more light opacifying pigments, such as titanium dioxide, zinc oxide, carbon black, cadmium sulfide, cadmium selenide, chromium oxide, iron oxide, lead oxide, azo pigments, anthraquinones, phthalocyanines, tetrachloroisoindolinones, quinacridones, isoindolines, and perylenes, pyrrolopyrroles.

The delayed release oral dosage form of the invention can further comprises fillers, binders, disintegrants, lubricants, flow agents, plasticizers and mixtures thereof. The dosage form can also comprise an extra-granular phase comprising one or more active ingredients, one or more excipients selected from the group consisting of fillers, binders, disintegrants, adhesives, wetting agents, and adjuvants, and mixture thereof.

The delayed release oral dosage form of the invention can be prepared using a method of comprising the steps of:
(a) obtaining, through heating or by dissolution in a suitable medium, a liquid form of said one or more excipients;
(b) adding from 0% to 100% of said polymers or copolymers to the liquid form of said one or more excipients, thereby producing a granulating liquid;
(c) mixing said one or more active ingredient with the remainder of said polymers or copolymers, thereby producing a mixture;
(d) granulating said mixture with said granulating liquid so as to obtain granules;
(e) optionally, blending said granules with other active ingredients and excipients, thereby obtaining a final mixture;
(f) encapsulating said granules or said final mixture or compressing said granules or said final mixture into a tablet.

Furthermore, a coating can be applied to the granules and/or to the compressed tablet so prepared.

### DETAILED DESCRIPTION OF THE DRAWINGS

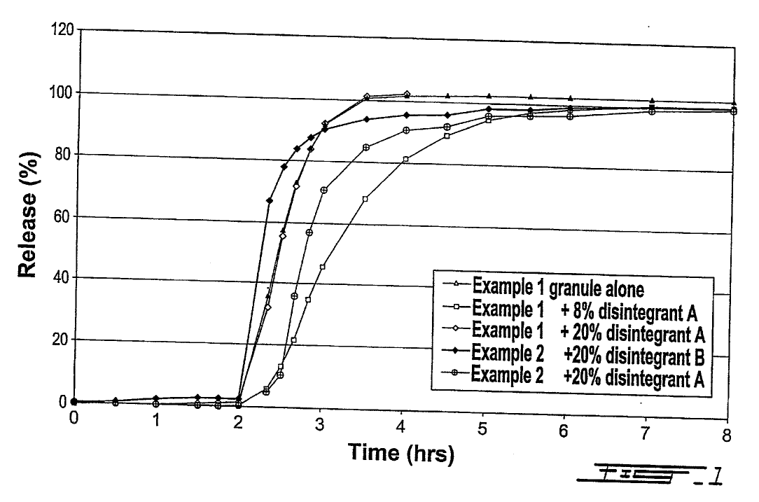

FIG. 1 illustrates the release of diclofenac-Na in vitro from tablets according to examples 1 and 2 of the present invention,

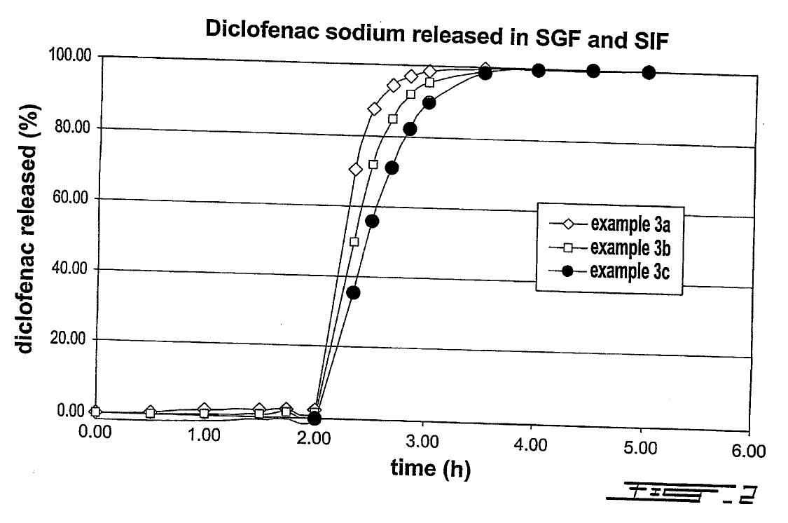

FIG. 2 illustrates the release of diclofenac-Na in vitro from tablets according to examples 3a, 3b and 3c of the present invention.

All tablets were kept for two hours in simulated gastric fluid and later transferred to simulated intestinal fluid using a USP apparatus II at 200 rpm.

### DETAILED DESCRIPTION OF THE INVENTION

The present invention provides a novel oral dosage form vehicle which can essentially behave as an enterically coated dosage form without the need for the formulation and application of an enteric coating. Although the dosage form can transport a whole range of active ingredients, the dosage is particularly suitable for products containing non-steroidal anti-inflammatory drugs (NSAID).

The products according to the invention are characterized by a high stability in acidic media and a rapid release of active ingredient at increased pH. The release can be controlled by the size and composition of the granules. The procedure for manufacturing products according to the invention is simple and cost efficient when compared to manufacture of the conventional enterically-coated products.

The present invention relates to a delayed release pharmaceutical oral dosage form and method of making same. The delayed release dosage form is preferably a tablet. The delayed release dosage form is characterized by a mixture of one or more active ingredients and one or more excipients selected from the group of fatty alcohols, fatty acid esters, natural or synthetic waxes and pH dependent soluble polymers.

Thus, in one aspect, the present invention provides oral dosage forms, i.e. granules, which are tableted in oral dosage forms. The active ingredient is preferably an NSAID. The active ingredient is essentially uniformly distributed in a mass of one or more excipients selected from the group of solid aliphatic alcohols, mixtures of esters of saturated fatty alcohols and saturated fatty acids or natural or synthetic waxes, hydrogenated castor oil, hydrogenated vegetable oil, gums, or mixtures thereof. The granules also comprise acrylic and methacrylic acid polymers and copolymers exhibiting a pH-dependent solubility.

In one specific embodiment, the invention discloses that particularly advantageous preparations can be obtained by adding pH dependent polymers to a molten fatty alcohol and use this mixture for granulating the active ingredient. This technique is referred to as melt granulation. This melt granulation is first conducted in a temperature range suitable for the material to be melted, which is commonly between about 50 to 60° C., followed by cooling to about room temperature.

The above described melt granulation procedure provides an essentially homogeneous preparation where the active is entrapped in waxy granules containing a pH-dependent component. The hydrophobic character of the waxy component ensure that the active ingredient remains essentially undissolved during its passage through the stomach and provide for a rapid opening of the granules at higher pH values.

The prepared granules are advantageously passed through appropriate sieves in order to obtain granules having a diameter less than 850 microns.

Further common excipients, that may be used to improve binding, disintegration and/or lubrification, may be added and are known to those of ordinary skill in the art.

In another specific embodiment, the invention relates to preparations in which:
i) one or more active ingredient(s) is uniformly distributed in a mass composed of a mixture comprising at least one pH-dependent component and at least one saturated fatty alcohol previously dissolved in ethylic alcohol to obtain a clear granulating liquid;
ii) the procedure consists in granulating the homogeneously mixed active ingredient and polymeric component using the previously prepared granulating liquid, without heating or cooling;
iii) the resulting preparation is then passed through appropriate sieves in order to obtain granules having a diameter less than about 1000 microns and preferably less than about 850 microns;
iv) the obtained granulated material can be used in association with one or more additional active ingredients comprised in an external (or extra-granular) excipient phase consisting of fillers, binders, disintegrants, adjuvants, etc. to obtain tablets.

This approach represents a new melt granulation procedure. Using a combination of pH-dependent polymeric materials with excipients containing a hydrophobic segment in their structure, the active ingredient may be entrapped into a gastric insoluble composition that can ensure its delayed release. In contrast with the conventional enteric coating procedures providing particles, granules or beads where the active is surrounded by a film or membrane obtained from a polymeric solution or dispersion, the preparation of the invention produces an enteric protection in the absence of an actual enteric coating.

The present invention is also distinct from the classical melt granulation procedures known in the prior art. In contrast with these prior art procedures, the active admixture with suitable excipients will lead to a granulated active that can be compressed or encapsulated together with one or more additional active ingredients.

Advantageously, light-protective, or light-opacifying, pigment or flakes may also be incorporated in the melt granulation method so as to impart light-protective properties to the resulting granules. This avoids the need for a light-protective coating to be placed onto the compressed dosage form. Organic or inorganic pigments may be advantageously used in accordance with the present invention. The pigments may be incorporated at various stages of the melt granulation, for example in the powder blend or the granulating liquid solution.

Examples of inorganic pigments are titanium dioxide, zinc oxide, carbon black, cadmium sulfide, cadmium selenide, chromium oxide, iron oxide, lead oxide and the like. The preferred light protective pigment is titanium dioxide.

Examples of organic pigments are azo pigments, anthraquinones, phthalocyanines, tetrachloroisoindolinones, quinacridones, isoindolines, perylenes, pyrrolopyrroles (such as Pigment Red 254) and the like.

It is particularly worthy of mention that the procedure of the invention can be used for a large number of pharmaceutical active molecules and will generate preparations having advantageous flowing, tableting, disintegration and dissolution properties.

In some embodiments of the present invention, the delayed release of the oral dosage form of the invention comprising the pH dependent material is obtained with a pharmaceutically acceptable acrylic and methacrylic polymers and co-polymers. Non limiting examples of such polymers and co-polymers are acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

It has now been discovered and is an aspect of this invention that polymers from methacrylates group, particularly anionic polymer containing carboxylic functions, may be included in the melt granulation process.

In preferred embodiments, the acrylic polymer is comprised of one or more anionic methacrylate copolymers which are well known in the art, and are described as fully polymerized copolymers of acrylic and methacrylic acid esters with a certain content of carboxylic groups. Such pH-dependent polymers constitute in total about 5% to about 50%, preferably about 10% to about 30%, and more preferably about 12% to about 20%, of the total weight of the composition. In order to obtain a desirable dissolution profile, it may be necessary to incorporate various amounts of methacrylic acid copolymers having differing properties, such as different amount of carboxylic functional groups able to protonate/deprotonate depending on the pH changes.

It has been furthermore discovered that certain methacrylic acid ester-type polymers that are generally used for preparing pH-dependent coatings, may be used as pH dependent materials in the present invention. For example, there are a family of copolymers synthesized from diethylaminoethyl methacrylate and other neutral methacrylic esters, also known as methacrylic acid copolymer or polymeric methacrylates, commercially available as Eudragit®.RTM. from Rohm Pharma.

More generally, the pH-dependent polymers and copolymers typically used in enteric coatings can be used as pH-dependent polymers and copolymers in the present invention. For example, such a coating can typically comprise a mixture of two acrylic resin lacquers commercially available from Rohm Pharma under the trade-names Eudragit RTM RL30D® and Eudragit RTM RS30D®. These products are copolymers of acrylic and methacrylic esters with a low content of quaternary ammonium groups, the molar ratio of ammonium groups to the remaining neutral (meth)acrylic esters. In these typical coatings, the Eudragit RTM RL/RS® dispersions may be mixed together in any desired ratio in order to ultimately obtain a controlled-release formulation having a desirable dissolution profile. Desirable controlled-release formulations may be obtained, for instance, from a retardant coating derived from 100% Eudragit RTM RL®, 50% Eudragit RTM RL® and 50% Eudragit RTM RS®, and 10% Eudragit RTM RL and 90% Eudragit RTM RS. Of course, one skilled in the art will recognize that other acrylic polymers may also be used, such as, for example, Eudragit RTM L@.

Preferred oral dosage forms of the present invention may further comprise one or more pharmaceutically acceptable excipients selected from the group consisting of binding agents, disintegrants, adhesives and wetting agents, More preferably, such compositions are in the form of multiparticulate compositions, particularly compressed in matrix tablets, compositions particularly resistant to gastric fluid and exhibiting an immediate release of drug starting with pH 5.5.

Oral dosage forms of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; been wax, gelatin; glucose; starches such as, but not limited to, pregelatinized starches (e.g., National® 1511 and National® 1500); celluloses such as, but not limited to, methylcellulose; alginic acid and salts of alginic acid; PEG; guar gum; polysaccharide acids; povidone, for example povidone K-15®, K-30® and K-29/32®; and ethylcellulose (e.g., Ethocel®) and more preferred ones from the class of fatty alcohol, fatty acid ester, natural or synthetic waxes. Such binding agents and/or adhesives, if present, constitute in total about 5% to about 60%, preferably about 10% to about 50%, and more preferably about 12% to about 50%, of the total weight of the composition.

Oral dosage forms of the invention optionally comprise also one or more pharmaceutically acceptable disintegrants as excipients, particularly for tablet formulations. Suitable disintegrants include, either individually or in combination, starches, including sodium starch glycolate (e.g., Explotab® of PenWest) and pregelatinized corn starches (e.g., National® 1551, National® 1550, and Colorcon® 1500), celluloses such as purified cellulose, microcrystalline cellulose, methylcellulose, carboxymethylcellulose and sodium carboxymethylcellulose, croscarmellose sodium (e.g., Ac-Di-Sol® of FMC), alginates, crospovidone, and gums such as agar, guar. The preferred disintegrant is crosscarmelose in total about 5% to about 30%, preferably about 7% to about 25%, and more preferably about 8% to about 20%, of the total weight of the composition.

The oral dosage forms of the present invention could be in the form of tablets or other forms such as, for example, granule-containing capsules.

The tablets may also be coated with suitable coatings, such as, for example, anti-sticking coatings or color coatings, to impart desirable properties to the outside of the tablets. Also, other standard enteric coating materials may be used for example phthalates, e.g. cellulose acetate phthalate or preferably hydroxypropylacetate phthalate or polyvinylacetate phthalate. Furthermore, mixtures of these and other materials may be used to produce coated granules.

### Examples of Tablet Design

Two different approaches were considered, a first approach with delayed release characteristics being designed at granule level and a second approach designed at entire tablet level.

A. Delayed release properties are ensured at granule level: multiparticulates where obtained from a preferred composition having delayed release characteristics

### Example 1

The active ingredient (Diclofenac Sodium) and the pH-dependent polymer were placed in a jacketed bowl and mixed for homogenisation. The granulation liquid was obtained by heating the fatty alcohol at 55° C. The granulating liquid was incorporated into the mixed powders and the granulation process was continued until granulation occurred. The granulated material was then transferred to a metal tray, and cooled to 22-24° C. An appropriate grinder was then used to mill the granulated material. The milled material was then successively screened through a 2 mm and a 0.850 mm screen. The granules were mixed with an appropriate amount of disintegrant and the resultant composition was compressed into tablets of 625 mg weight having a diameter of 8.5 mm using a single punch press. The final formulation expressed as weight percentages contained 70% disintegrant and 30% of granulates (composition breakdown: 12% active, 15% fatty alcohol and 3% methacrylic polymer). The tablets were then subjected to dissolution testing in accordance with the procedure mentioned before (USP apparatus I, 200 rpm, 2h SGF, 2h SIF).

The results are shown in FIG. 1 (example 1).

### Example 2

The fatty alcohol was melted and preliminary mixed with a methacrylic copolymer under vigorous stirring to obtain a homogeneous dispersion.

The Diclofenac Sodium was placed in a jacketed bowl and the dispersion was added gradually, under continuous stirring, until granulation occurred. The granulated material was then transferred to a metal tray, and cooled to 22-24° C. An appropriate grinder was then used to mill the granulated material. The milled material was then successively screened through a 2 mm and a 0.85 mm screen. The granules were then mixed with an appropriate amount of disintegrant (representing 8 to 20% from the total mass of the diclofenac layer) and the resultant mixture was compressed into tablets. For this preparation, the diclofenac layer weight could vary from 215 to 250 mg.

Tablets of Example 2 were then subjected to dissolution testing in accordance with the procedure mentioned before [USP apparatus I, 200 rpm, 2h Simulated Gastric Fluid (SGF), 2h Simulated Intestinal Fluid (SIF)].

The results are shown in FIG. 1 (example 2).

FIG. 1 shows also the influence of disintegrant type and proportion employed together with the two preparations described in Examples 1 and 2 in order to induce tablet disintegration.

### Example 3

The Diclofenac Sodium and a fraction of the pH-dependent polymer (30% from total quantity) were placed in a bowl and mixed for homogenisation. The fatty alcohol was dissolved in ethanol and the rest of the polymer (70%) was incorporated to obtain the granulation liquid.

The granulation process was conducted without heating jacket, using the mixer at 500 rpm and the chopper at 1200 rpm until granulation occurred. The agglomerates were then broken down by any suitable means to comminute oversize agglomerates and produce a mixture of powder and small particles preferably with a diameter under 0.85 mm. An appropriate amount of disintegrant was added and the resultant mixture was compressed into tablets.

The tablets of Example 3 were then subjected to three dissolution tests, Examples 3a, 3b and 3c, in accordance with the procedure mentioned in example 1.

The dissolution profiles 3a, 3b and 3c, shown in FIG. 2, indicate a modulation of opening times of diclofenac granules in SIF in relationship with the ratio active/pH dependent polymer/binder. By keeping the disintegrant type and proportion constant in all three preparations, the composition of granules was shown to be variable at will (table 1)

TABLE I
Example 3 (granules composition)

| Ingredients | Example 3a | Example 3b | Example 3c |
|---|---|---|---|
| Active | 41.3 | 41.0 | 33.0 |
| principle | | | |
| Aliphatic | 12.2 | 18.0 | 23.3 |
| alcohol | | | |
| (binder) | | | |
| pH-dependent | 46.5 | 41.0 | 43.7 |
| polymer | | | |
| Final tablet | +8% disintegrant | +8% disintegrant | +8% disintegrant |
| (mg) | B | B | B |
| | 200 mg | 200 mg | 250 mg |

B. Delayed release properties are ensured by entire tablet: a preferred composition having delayed release characteristics is obtained by a melt granulation process and provides a protection for the passage through the gastric segment by the limited capacity of the tablet to hydrate in acidic medium. By keeping its integrity, the tablet is able to ensure delayed characteristics.

### Example 4

The fatty alcohol was first melted. The Sodium Diclofenac and a methacrylic copolymer were placed in a jacketed bowl and the granulated liquid was added gradually under continuous mixing until granulation occurred. The granulated material was then transferred to a metal tray, and cooled to 22-24° C. An appropriate grinder was then used to mill the granulated material. The milled material was then successively screened through a 2 mm and a 0.85 mm screen. The granules were then compressed into tablets which were tested in conditions mentioned before (2h SGF followed by SIF, 200 rpm, UPS apparatus II). The delayed release properties are more pronounced when the matrix approach is used, the dissolution profiles showing a complete drug release after more than 10 h.

### C. Light Protection of Granules by Incorporation of One or More Pigments or Flakes

### Example 5

The active ingredient Sodium Diclofenac and the melted polymer (granulating liquid) were placed in a jacketed bowl and mixed homogeneously. The granulating liquid was obtained by adding at least one pigment powder to the fatty alcohol solution prepared ahead of time using ethanol and eventually heated to 55° C. to induce melting. The liquid was gradually dispersed under continuous mixing until granulation occurred. The preparation was transferred to a metal tray, cooled to 22-24° C. and then successively screened through a 2 mm and a 0.85 mm screen. A final admixture was obtained by mixing the granulated active with a disintegrant. The resultant preparation was compressed in tablets where the diclofenac layer weight could vary from 200 to 300 mg. The amount of disintegrant was selected to represent about 5 to about 35 wt % of the active ingredient present in the tablet.

### Examples 6-8

Granules were made as shown above in example 5 with varying amounts of pigments. More specifically titanium dioxide was set at 4 wt %, 8 wt % and 15 wt % based on the total mass of the granules. Mixtures of titanium dioxide and flakes (red #40 lake) were also used to provide opacity to the granulated active.

### D. Tablet Testing

Desired tap and bulk densities of the granulation are normally about 0.3 g/ml to about 1.0 g/ml. Tablets friability preferably is less than about 1.0%, more preferably less than 0.8%, and still more preferably less than about 0.5%, in a standard test.

It is known that several factors influence the dissolution, in a solvent medium, of a drug from its carrier, These factors include the surface area of the drug presented to the solvent medium, the solubility of the drug in the solvent medium, and the driving forces of the saturation concentration of dissolved materials in the solvent medium.

A composition having a dissolution profile in which substantially less than 5% of the drug contained therein is released in the first two hours after placement in a SGF dissolution medium is considered to be a delayed-release composition. In contrast, immediate-release compositions typically release at least 50% of the drug contained therein in the first hour after placement in a dissolution medium.

The tablets in accordance with one embodiment of the invention show about 1% to about 3 (or 5) % dissolution in 2 hours in SGF, about 30% to about 70% dissolution in the first half-hour in SIF, and at least about 90% dissolution in the first hour in SIF. Preferred tablets of the invention show about 0.5% to about 2.5% dissolution in 2 hours in SGF, about 60% to about 80% dissolution in the first 30 min in SIF, and at least about 95% dissolution in 1 hour in SIF. Most preferred tablets of the present invention show about 1.5% to about 2.5% dissolution in 2 hours in SGF, about 75% to 85% dissolution in the first 30 min in SIF, and substantially complete dissolution in one hour in SIF.

1. A delayed release oral dosage form comprising one or more active ingredients within a granulated composition, which further comprises:
a) one or more excipients selected from the group of solid aliphatic alcohols, fatty acid esters, mixtures of esters of saturated fatty alcohols and saturated fatty acids, natural waxes, synthetic waxes, hydrogenated castor oil, hydrogenated vegetable oil, gums, and mixtures thereof; and
b) one or more polymers or copolymers exhibiting a pH-dependent solubility.

2. The delayed release oral dosage form of claim 1 further comprising an extra-granular phase comprising one or more active ingredients, one or more excipients selected from the group consisting of fillers, binders, disintegrants, adhesives, wetting agents, and adjuvants, and mixture thereof.

3. The delayed release oral dosage form of claim 1, wherein the dosage form is a monolithic dosage form.

4. The delayed release oral dosage form of claim 1, wherein the dosage form is a compressed tablet.

5. The delayed release oral dosage form of claim 4, wherein the compressed tablet is coated by a coating.

6. The delayed release oral dosage form of claim 1, wherein the dosage form is a capsule containing the granulated composition.

7. The delayed release oral dosage form of claim 1, wherein the one or more polymers or copolymers are acrylic and methacrylic acid polymers and copolymers.

8. The delayed release oral dosage form of claim 1, wherein the one or more polymers or copolymers are selected from the group consisting of acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

9. The delayed release oral dosage form of claim 1, wherein the one or more polymers or copolymers is a methacrylic polymer.

10. The delayed release oral dosage form of claim 1, wherein the one or more excipients is a fatty alcohol.

11. The delayed release oral dosage form of claim 1, wherein the one or more excipients is a saturated fatty alcohol.

12. The delayed release oral dosage form of claim 1, wherein said one or more excipients is a solid aliphatic alcohol.

13. The delayed release oral dosage form of claim 1, wherein said one or more active ingredients in selected from an NSAID, a prostaglandin and mixtures thereof.

14. The delayed release oral dosage form of claim 1, wherein said granulated composition further comprises one or more light opacifying pigments.

15. The delayed release oral dosage form of claim 14, wherein said light opacifying pigment is selected from the group consisting of titanium dioxide, zinc oxide, carbon black, cadmium sulfide, cadmium selenide, chromium oxide, iron oxide, lead oxide, azo pigments, anthraquinones, phthalocyanines, tetrachloroisoindolinones, quinacridones, isoindolines, and perylenes, pyrrolopyrroles.

16. The delayed release oral dosage form of claim 15, wherein said light opacifying pigment is titanium dioxide.

17. The delayed release oral dosage form of claim 1, wherein said granulated composition further comprises fillers, binders, disintegrants, lubricants, flow agents, plasticizers and mixtures thereof.

18. The delayed release oral dosage form of claim 1, wherein the granulated composition is coated by a coating.

19. The delayed release oral dosage form of claim 2, wherein the extra-granular phase comprises crosscarmelose.

20. The delayed release oral dosage form of claim 1, wherein the granulated composition comprises granules having a diameter less than about 1000 microns.

21. The delayed release oral dosage form of claim 20, wherein the granules have a diameter less than about 850 microns.

22. A delayed release oral dosage form comprising one or more active ingredients essentially uniformly distributed in a granulated composition comprising:
a) one or more excipients selected from the group of solid aliphatic alcohols, mixtures of esters of saturated fatty alcohols and saturated fatty acids or natural or synthetic waxes, hydrogenated castor oil, hydrogenated vegetable oil, gums, or mixtures thereof;
b) acrylic and methacrylic acid polymers and copolymers exhibiting a pH-dependent solubility;
c) one or more light opacifying pigments; and
d) an extra-granular phase comprising a disintegrant.

23. A method of making a delayed release oral dosage form as described in claim 1 comprising:
a) obtaining, through heating or by dissolution in a suitable medium, a liquid form of said one or more excipients;
b) adding from 0% to 100% of said polymers or copolymers to the liquid form of said one or more excipients, thereby producing a granulating liquid;
c) mixing said one or more active ingredient with the remainder of said polymers or copolymers, thereby producing a mixture;
d) granulating said mixture with said granulating liquid so as to obtain granules;
e) optionally, blending said granules with other active ingredients and excipients, thereby obtaining a final mixture; and
f) encapsulating said granules or said final mixture or compressing said granules or said final mixture into a tablet.

24. A method of making a delayed release oral dosage form as described in claim 1 comprising:
a) obtaining, through heating or by dissolution in a suitable medium, a liquid form of said one or more excipients;
b) adding from 0% to 100% of said polymers or copolymers to the liquid form of said one or more excipients, thereby producing a granulating liquid;
c) mixing said one or more active ingredient with the remainder of said polymers or copolymers, thereby producing a mixture;
d) blending said mixture with said granulating liquid so as to obtain a pourable blend;
e) pouring said blend onto a solidification substrate;
f) obtaining the solidification of said blend on said substrate;
g) removing the solidified blend from said substrate;
h) milling the solidified blend into granules; and
i) encapsulating said granules or compressing said granules into a tablet shape.

25. The method of claim 24 further comprising before said encapsulating or compressing step, the step of applying a coating to the granules.

26. The method of claim 24, wherein step i) comprises compressing said granules into a tablet shape and further comprises applying a coating to the tablet.

27. A delayed release dosage form produced by the process of claim 23.
